(19) United States
(12) Patent Application Publication
(10) Pub. No.: US 2014/0194694 A1
(43) Pub. Date: Jul. 10, 2014

(54) Tracheal Intubation Device

(71) Applicant: Tien-Sheng Chen, Taipei City (TW)

(72) Inventor: Tien-Sheng Chen, Taipei City (TW)

(21) Appl. No.: 14/210,580

(22) Filed: Mar. 14, 2014

Related U.S. Application Data
(63) Continuation-in-part of application No. 13/354,388, filed on Jan. 20, 2012, now Pat. No. 8,721,535.

(30) Foreign Application Priority Data
Sep. 9, 2011 (TW) 100132692

(51) Int. Cl. A61B 1/05; A61B 1/00; A61M 16/04; A61B 1/267

(52) U.S. Cl. CPC A61B 1/053; A61B 1/267; A61B 1/00052; A61M 16/0488; A61B 1/00004; A61B 1/0002; A61B 1/00016; A61B 1/00066
USPC 600/188

(57) ABSTRACT

A tracheal intubation device for placing an endotracheal tube in the trachea of a patient includes a bar-shaped camera, a handle portion, a guiding portion, and a display device. The bar-shaped camera for capturing images is placed inside the endotracheal tube. The display device is electrically connected to the bar-shaped camera for displaying images captured by the bar-shaped camera. The guiding portion connected to the handle portion has a front end and a rear end. The rear end is connected to the handle portion, and the bar-shaped camera and the endotracheal tube are movably situated in the guiding portion.

### CROSS REFERENCE

The present application is a continuation of U.S. application Ser. No. 13/354,388 filed on Jan. 20, 2012.

### BACKGROUND OF THE INVENTION

1. Field of the Invention

The present invention relates to a medical device. More specifically, the present invention relates to a tracheal intubation device for placing an endotracheal tube into the trachea of a patient.

2. Description of the Related Art

For apnea patients, tracheal intubation is an essential operation for sustaining respiration to maintain the vital signs of a patient. Medical personnel have to place an endotracheal tube into the trachea of a patient to ensure that oxygen is provided in a short period of time. Therefore, it is an important task for medical personnel to place the endotracheal tube efficiently. In practice, tracheal intubation devices equipped with a camera are often used by medical personnel to confirm the location of the trachea of a patient and then to place the endotracheal tube into the trachea of the patient. Tracheal intubation devices disclosed in U.S. Pat. No. 4,306,547 and U.S. Patent Publication Number 2007/0106121 are the most widely used tracheal intubation devices, and the image capturing device of each tracheal intubation device is installed on the lateral side of the endotracheal tube slot. When the glottis of the trachea of the patient is observed via the image capturing device, medical personnel push the endotracheal tube installed on the lateral side of the image capturing device into the trachea of the patient.

Although the tracheal intubation device disclosed in US Patent Number 2007/0106121 does improve the process of tracheal intubation, some drawbacks during operation of the device need to be eliminated. Sometimes, due to the physical structure of a patient, the visual field of this kind of tracheal intubation device is blocked by the parallax caused by the camera installed on the lateral side of the endotracheal tube slot, or the endotracheal tube. Consequently, since the glottis of the trachea cannot be observed, the endotracheal tube cannot be precisely placed into the trachea of a patient by medical personnel. The Acta Anaethesiologica Scandinavica published in 2010 (2010;54(9):1050-61) also indicates that this widely used tracheal intubation devices may occasionally fail in precise placement.

In view of the fact that efficient intubation is critical for the survival of a patient, there is a need to provide an intubation device by which intubation can be performed easily and precisely.

### SUMMARY OF THE INVENTION

It is an object of the present invention to provide a tracheal intubation device by which an intubation procedure can be operated easily and precisely.

To achieve the abovementioned object, the tracheal intubation device of the present invention comprises a bar-shaped camera, a handle portion, a guiding portion, and a display device. The bar-shaped camera is placed inside an endotracheal tube for capturing an image. The display device is connected with the handle portion and electrically connected to the bar-shaped camera for displaying the image captured by the bar-shaped camera. The guiding portion is connected to the handle portion and comprising a front end, a rear end, a tongue piece, a barrier and a slot portion. The tongue piece is extended form the rear end to the front end. The barrier is extended form the rear end to the front end and substantially perpendicular to the tongue piece. The slot portion comprises a main surface portion connected with the barrier and the main surface portion is substantially paralleled to the tongue piece. The slot portion is connected with the front end, thus a restricted passage is formed by the tongue piece, the slot portion and the barrier. An open passage is formed by the tongue piece and the barrier, whereby, the endotracheal tube and the bar-shaped camera are movably placed within the open passage and the restricted passage.

The present invention further provides a tracheal intubation device of the comprising a bar-shaped camera, a handle portion, a guiding portion, and a display device. The bar-shaped camera is placed inside the endotracheal tube for capturing images. The display device is electrically connected to the bar-shaped camera for displaying images captured by the bar-shaped camera. The guiding portion is connected to the handle portion. The guiding portion comprises a front end and a rear end, and the rear end of the guiding portion is connected to the handle portion. The bar-shaped camera and the endotracheal tube are movably situated in the guiding portion.

According to one embodiment of the present invention, the tracheal intubation device of the present invention further comprises an insertion unit mounted on the bar-shaped camera for placing inside the endotracheal tube to allow the bar-shaped camera to move in synchrony with the endotracheal tube.

According to another embodiment of the present invention, the bar-shaped camera is made of a rigid material that allows the bar-shaped camera to guide the direction of movement of the endotracheal tube while the endotracheal tube is moving.

### BRIEF DESCRIPTION OF THE DRAWINGS

FIG. 1 is an exploded view illustrating the first embodiment of the tracheal intubation device of the present invention and the endotracheal tube.

FIG. 2 is a schematic drawing illustrating a usage state of the first embodiment of the tracheal intubation device of the present invention.

FIG. 3 is another schematic drawing illustrating the usage state of the first embodiment of the tracheal intubation device of the present invention.

FIG. 4 is an exploded view illustrating the second embodiment of the tracheal intubation device of the present invention and the endotracheal tube.

FIG. 5 is an exploded view illustrating the third embodiment of the tracheal intubation device of the present invention and the endotracheal tube.

FIG. 6 is a structural drawing of the fourth embodiment of the tracheal intubation device of the present invention.

FIG. 7 is another exploded view illustrating the fourth embodiment of the tracheal intubation device of the present invention.

FIG. 8 is an exploded view illustrating the fifth embodiment of the tracheal intubation device of the present invention and the endotracheal tube.

FIG. 9 is a schematic drawing illustrating a usage state of the fifth embodiment of the tracheal intubation device of the present invention.

FIG. 10 is a sectional view according to A-A direction of FIG. 9.

FIG. 11 is a sectional view according to B-B direction of FIG. 9.

### DETAILED DESCRIPTION OF THE PREFERRED EMBODIMENTS

The advantages and innovative features of the invention will become more apparent from the following detailed descriptions when taken together with the accompanying drawings.

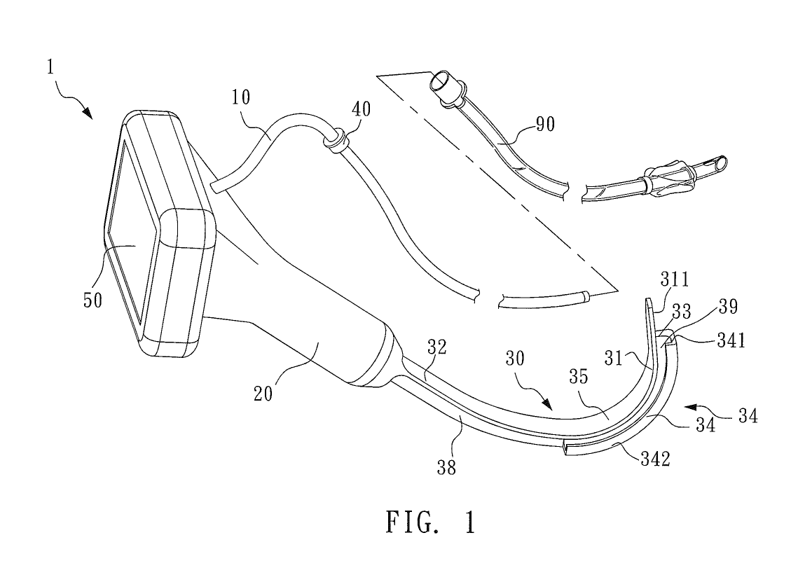

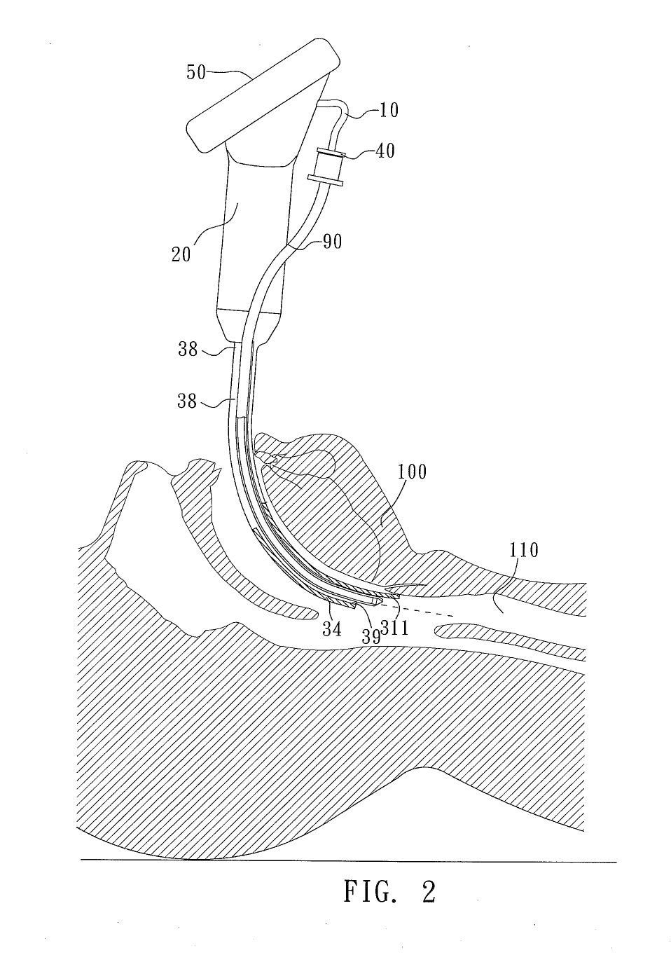

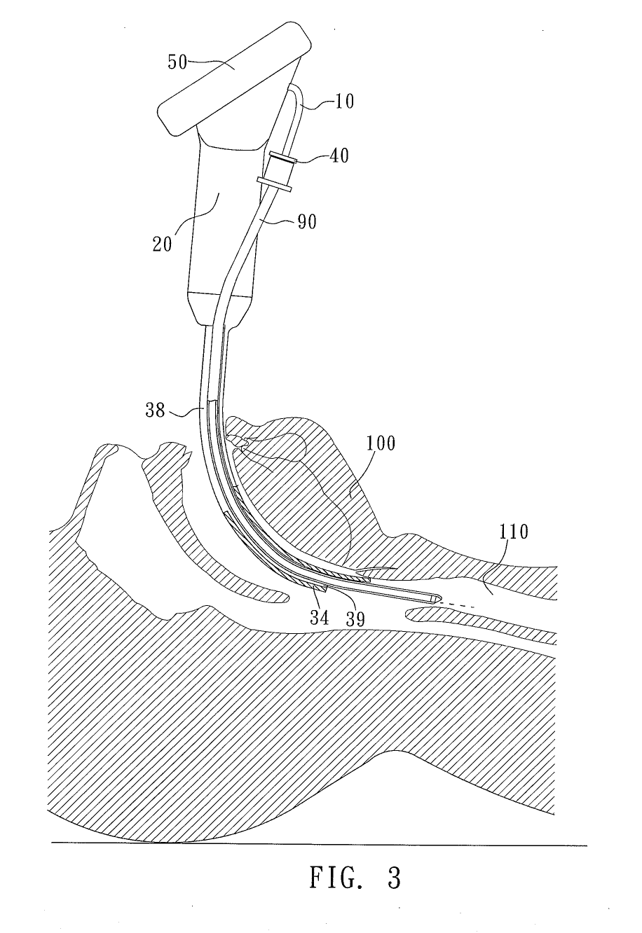

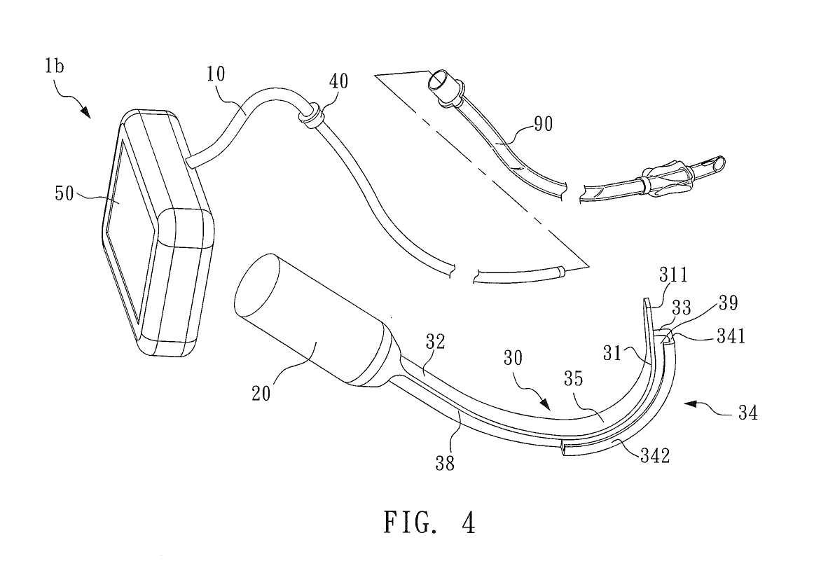

Please refer to FIG. 1 to FIG. 4, depicting embodiments of the tracheal intubation device of the present invention. FIG. 1 is an exploded view illustrating the first embodiment of the tracheal intubation device of the present invention and the endotracheal tube. FIG. 2 is a schematic drawing illustrating a usage state of the first embodiment of the tracheal intubation device of the present invention. FIG. 3 is another schematic drawing illustrating the usage state of the first embodiment of the tracheal intubation device of the present invention. FIG. 4 is an exploded view illustrating the second embodiment of the tracheal intubation device of the present invention and the endotracheal tube.

According to one embodiment of the present invention, as shown in FIG. 1, the tracheal intubation device 1 of the present invention comprises a bar-shaped camera 10, a handle portion 20, a guiding portion 30, an insertion unit 40, and a display device 50. The bar-shaped camera 10 is placed inside the endotracheal tube 90 to ensure that the endotracheal tube 90 can be precisely placed into the glottis of the trachea by reference to the image captured by the bar-shaped camera 10. The bar-shaped camera 10 is used for capturing images, such as an image of the glottis of the trachea of a patient. The display device 50 is electrically connected to the bar-shaped camera 10 for displaying the image captured by the bar-shaped camera 10.

As shown in FIG. 1, the guiding portion 30 is connected to the handle portion 20, and the guiding portion 30 comprises a front end 31, a rear end 32, a tongue piece 35, a barrier 33 and a slot portion 34. The rear end 32 of the guiding portion 30 is connected to the handle portion 20. The slot portion 34 is situated at the front end 31 of the guiding portion 30. The tongue piece 35 is extended form the rear end 32 to the front end 31. The barrier 33 is extended form the rear end 32 to the front end 31 and substantially perpendicular to the tongue piece 35. The slot portion 34 comprises a main surface portion 341 which is connected with the barrier 33 and substantially paralleled to the tongue piece 35. The slot portion 34 is connected with the front end 31, thus a restricted passage 39 is formed by the tongue piece 35, the slot portion 34 and the barrier 33. An open passage 38 is formed by the tongue piece 35 and the barrier 33; therefore, the endotracheal tube 90 and the bar-shaped camera 10 is movably placed within the open passage 38 and the restricted passage 39. As shown in FIG. 1, the slot portion 34 further comprises a restricted part 342 which is connected with the surface portion 341 and paralleled to the barrier 33 so that the slot portion 34 in the first embodiment is in U shape.

The bar-shaped camera 10 and the endotracheal tube 90 are movably situated in the open passage 38 and the restricted passage 39 of the guiding portion 30. It is noted that the front end 31 of the guiding portion 30 in this embodiment further comprises a protrusion part 311, and that the slot portion 34 does not extend to the protrusion part 311. The open passage 38 is formed in the rear end 32 of the guiding portion 30 to facilitate the placement of the endotracheal tube 90 and the bar-shaped camera 10 for entering the restricted passage 39 within the slot portion 34, as shown in FIG. 2 and FIG. 3. The insertion unit 40 is mounted on the bar-shaped camera 10 for placing inside the endotracheal tube 90 such that the bar-shaped camera 10 moves in synchrony with the endotracheal tube 90, as shown in FIG. 3, for ensuring that the position of the bar-shaped camera 10 relative to the endotracheal tube 90 remains unchanged during movement; i.e., the bar-shaped camera 10 always stays inside the endotracheal tube 90 while the endotracheal tube 90 is moving.

It is noted that the bar-shaped camera 10 is a soft tube in this embodiment. The front end of the bar-shaped camera 10 has a camera and an illuminating device such as an LED light bulb. The display device 50 is connected to the handle portion 20, but the present invention is not limited to this embodiment. As shown in FIG. 4, the display device 50 of the tracheal intubation device 1 b can be connected only to the bar-shaped camera 10 and detached from the handle portion 20. The shape of the guiding portion 30 is an arch curving from the rear end 32 to the front end 31, and the curve matches the curve of a human's oral cavity. The inner parts of the guiding portion 30 of this embodiment are an integrated form. The insertion unit 40 of this embodiment is a plastic plug, but the present invention is not limited to the abovementioned embodiment. The insertion unit 40 can be made of another material, and any device capable of placing the bar-shaped camera 10 inside the endotracheal tube 90 is applicable.

As shown in FIG. 2, when the tracheal intubation device 1 of the present invention is used, the guiding portion 30 is used for lifting the tongue base of the patient 100, and the bar-shaped camera 10 and the endotracheal tube 90 in the restricted passage 39 within the slot portion 34 enter the mouth of the patient 100. The protrusion part 311 of the guiding portion 30 also has guiding and orienting functions for allowing the bar-shaped camera 10 and the endotracheal tube 90 to move toward the glottis of the trachea precisely. Medical personnel only have to push the bar-shaped camera 10 and the endotracheal tube 90 into the throat of the patient 100 until the medical personnel observes the accurate location of the trachea 110 of the patient 100 via the display device 50. Then, the endotracheal tube 90 is pushed to detach the endotracheal tube 90 from the insertion unit 40. At which point, the endotracheal tube 90 is placed in the trachea 110.

It is noted that the tracheal intubation device 1 of the present invention not only can prevent the visual field of the glottis of the trachea from being misled due to the camera's parallax or being blocked by the inserted endotracheal tube but also can prevent damage to the upper respiratory tissue during intubation. Furthermore, because the bar-shaped camera 10 of the tracheal intubation device 1 of the present invention is placed inside the endotracheal tube 90, the volume of the guiding portion 30 of the tracheal intubation device 1 of the present invention can be reduced. Consequently, the guiding portion 30 can be placed into a mouth of the patient 100 easily, even in the case of patients with smaller mouths, shorter necks, or thicker tongues. Moreover, placing the bar-shaped camera 10 inside the endotracheal tube 90 allows the bar-shaped camera 10 to enter the trachea 110 together with the endotracheal tube 90 to ensure that the endotracheal tube 90 is placed into the trachea 110 precisely each time, without risk of failure.

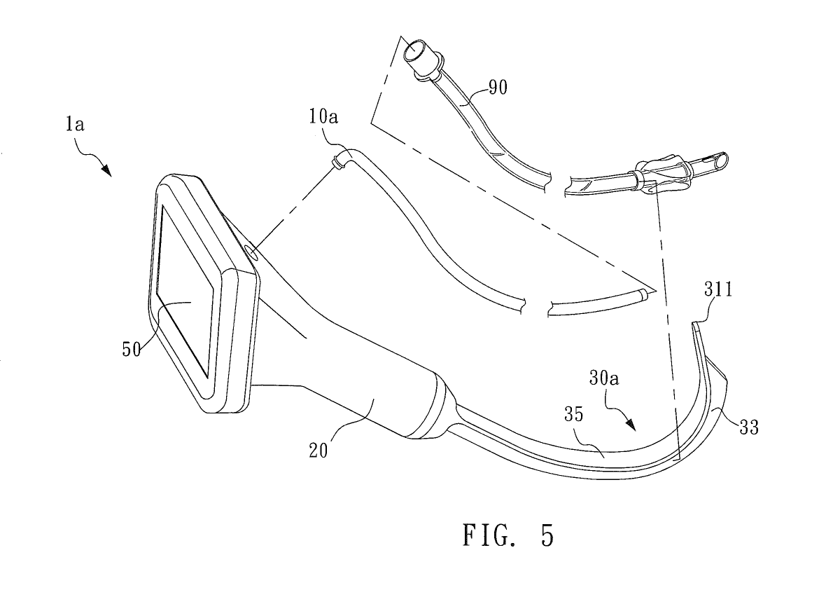

Please refer to FIG. 5, FIG. 5 is an exploded view illustrating the third embodiment of the tracheal intubation device of the present invention and the endotracheal tube.

The difference between the tracheal intubation device 1a of the third embodiment and the tracheal intubation device 1 is that there is no the surface portion 341 or restricted part 342 disposed the guiding portion 30a. The tracheal intubation device 1a only has a tongue piece 35 and a barrier 33. The tongue piece 35 is substantially perpendicular to the barrier 33. The bar-shaped camera 10a of the tracheal intubation device 1a in this embodiment is detachably connected to the handle portion 20, and the bar-shaped camera 10a is made of a rigid material that is curved to guide the direction of movement of the endotracheal tube 90 while it is moving. The bar-shaped camera 10 and the endotracheal tube 90 in this embodiment are accommodated within an area formed by the tongue piece 35 and the barrier 33, and the endotracheal tube 90 and the bar-shaped camera 10 can be moved within the area formed by the tongue piece 35 and the barrier 33. The usage and operating procedure of the tracheal intubation device 1a are the same as those of the tracheal intubation device 1. Therefore, the description is omitted. For such information, please refer to FIG. 2 and FIG. 3.

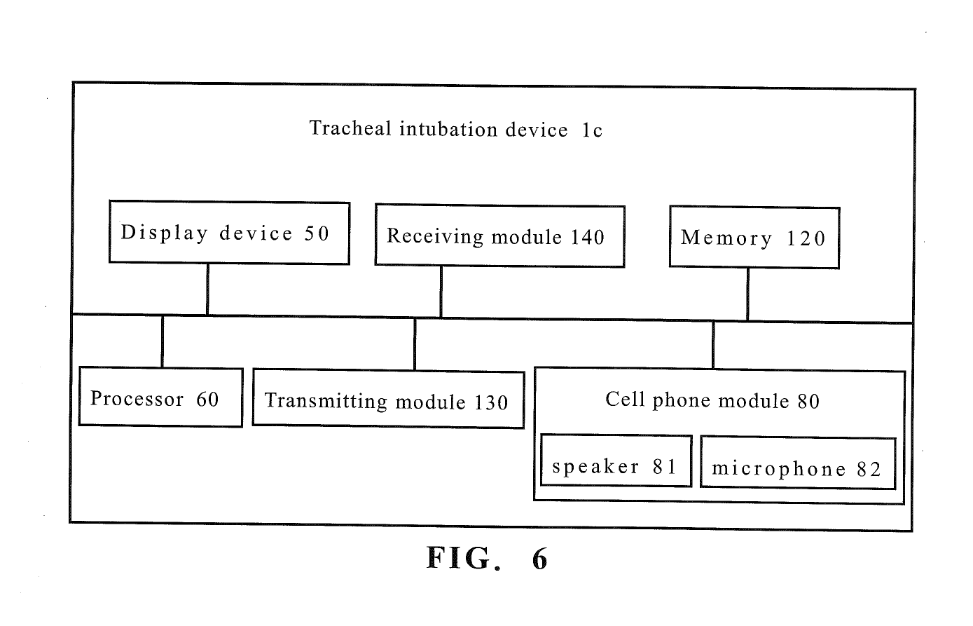

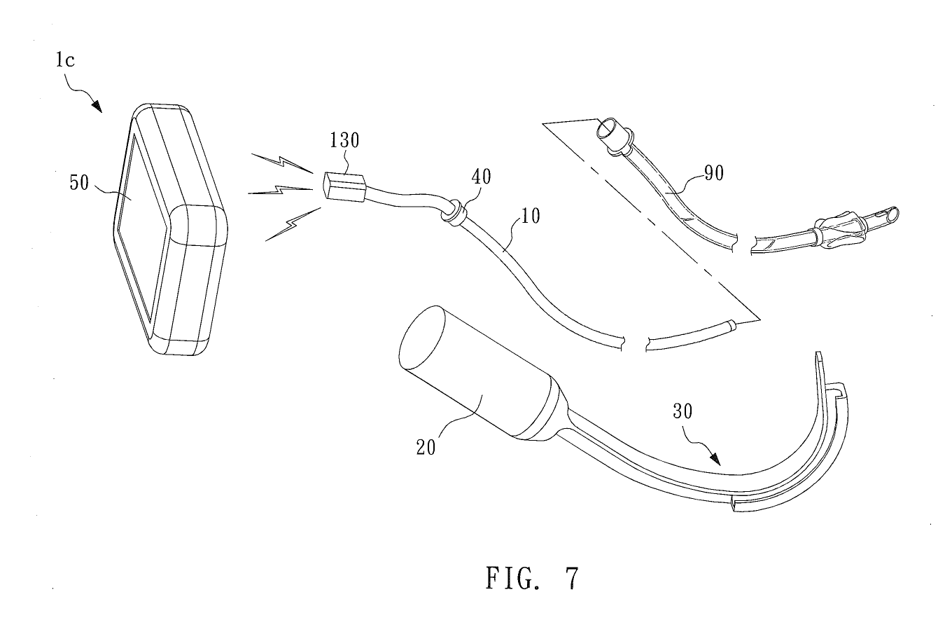

Please refer to FIG. 6 and FIG. 7, related to the fourth embodiment of the tracheal intubation device of the present invention. FIG. 6 is a structural drawing of another embodiment of the tracheal intubation device of the present invention. FIG. 7 is another exploded view illustrating the fourth embodiment of the tracheal intubation device of the present invention.

As shown in FIG. 6, the tracheal intubation device 1c of the present invention further comprises a processor 60, a receiving module 140, a cell phone module 80, a memory 120, and a transmitting module 130. The processor 60, the receiving module 140, the cell phone module 80, and the memory 120 are separately electrically connected to the display device 50. For example, electronic components like the processor 60 can be placed inside the shell of the display device 50, and the transmitting module 130 is connected to the bar-shaped camera 10. In this embodiment, the display device 50 is an individual display monitor. Images captured by the bar-shaped camera 10 are transmitted to the receiving module 140 via the transmitting module 130 and displayed on the display device 50 afterwards. Moreover, the cell phone module 80 comprises a speaker 81 and a microphone 82 for allowing medical personnel to talk to rescue personnel via the cell phone module 80. The memory 120 is used for storing images captured by the bar-shaped camera 10 and other essential control programs. The processor 60 is used for implementing the operations mentioned above. It is noted that the receiving module 140 and the transmitting module 130 both can be wireless communication devices employing a method such as Bluetooth, and the memory 120 can be a memory card. It is noted that the abovementioned devices and modules can also be installed in the tracheal intubation device 1, 1a, 1b or 1d of the present invention. However, the present invention is not limited to this embodiment.

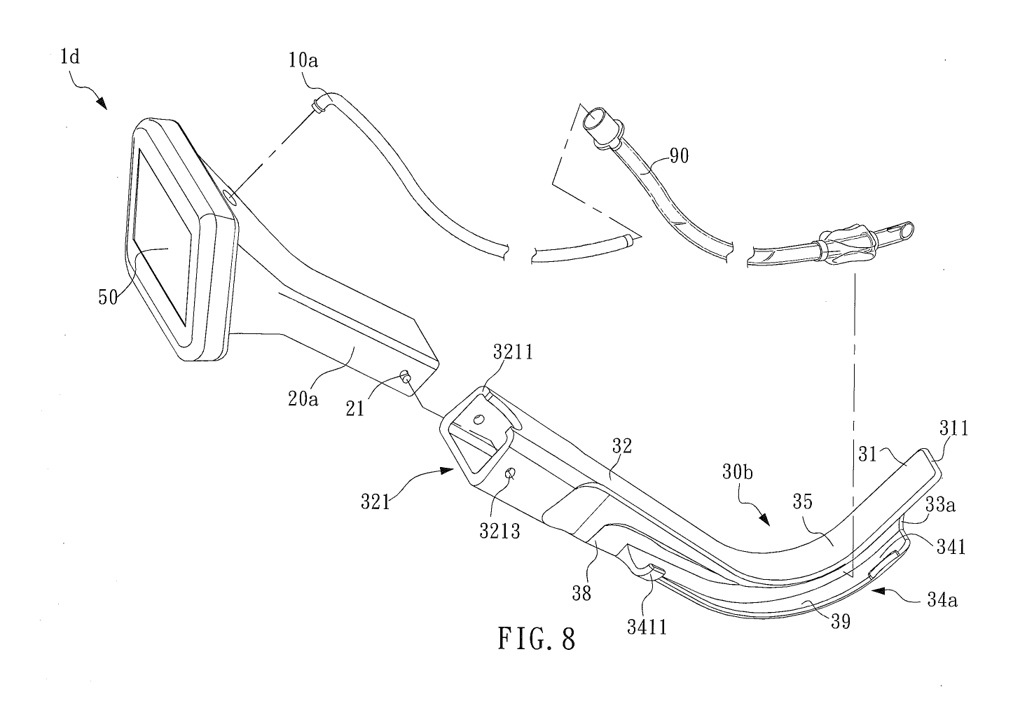

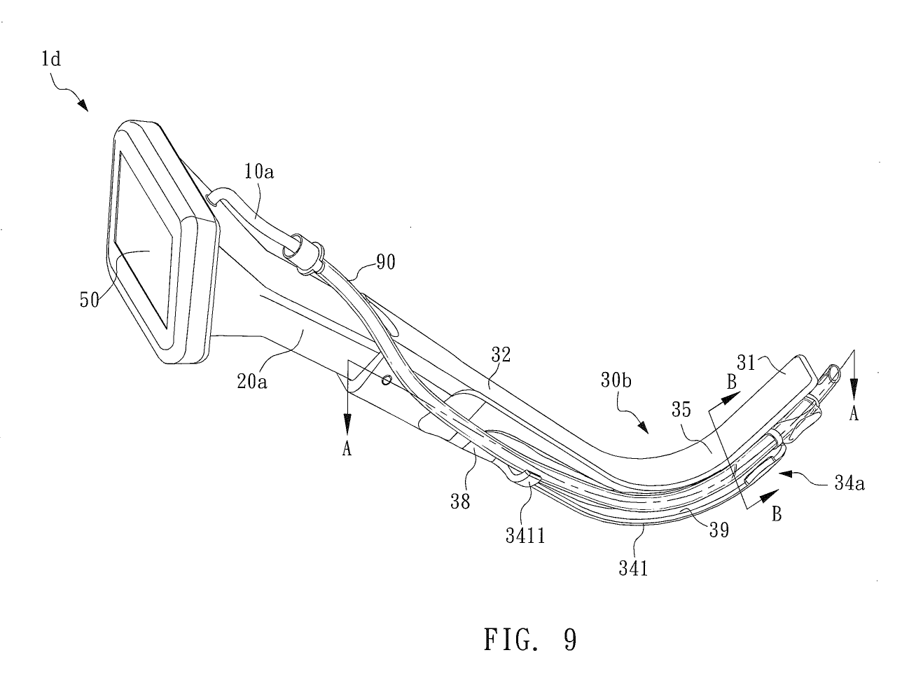

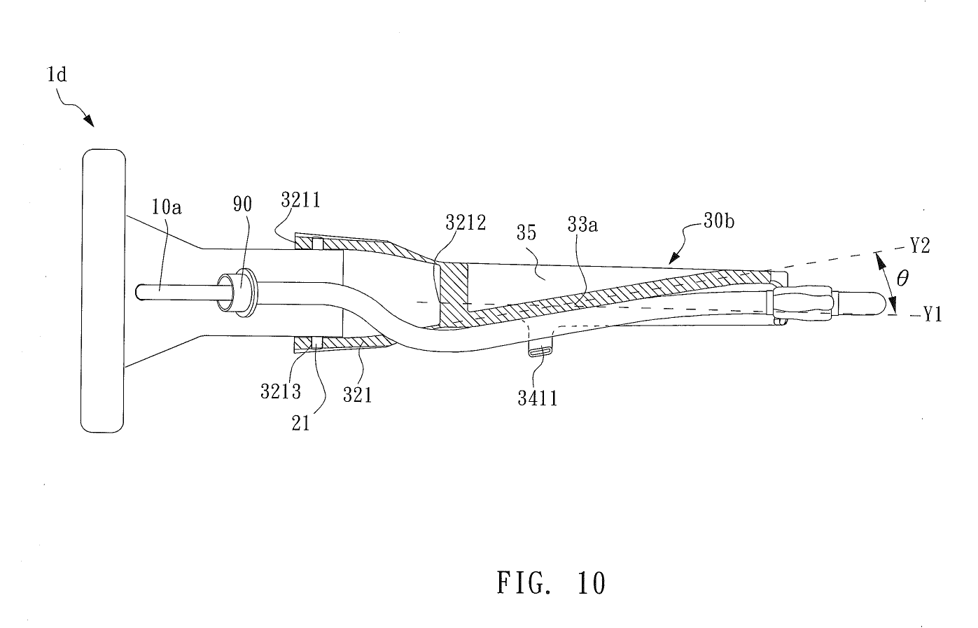

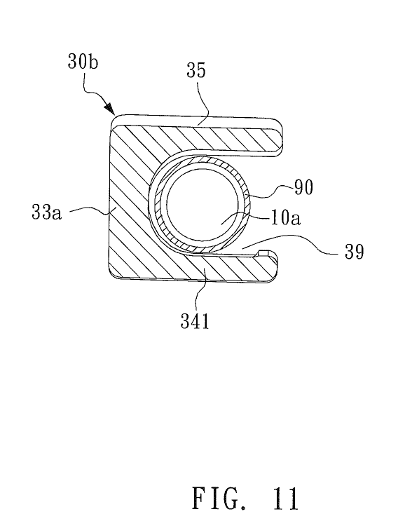

Please refer to FIG. 8 to FIG. 11, depicting embodiments of the tracheal intubation device of the present invention. FIG. 8 is an exploded view illustrating the fifth embodiment of the tracheal intubation device of the present invention and the endotracheal tube. FIG. 9 is a schematic drawing illustrating a usage state of the fifth embodiment of the tracheal intubation device of the present invention. FIG. 10 is a sectional view according to A-A direction of FIG. 9. FIG. 11 is a sectional view according to B-B direction of FIG. 9.

As shown in FIG. 8, according to the fifth embodiment of the present invention, the tracheal intubation device ld of the present invention comprises the bar-shaped camera 10a, the handle portion 20a, the guiding portion 30b, and the display device 50. The bar-shaped camera 10a is placed inside the endotracheal tube 90 to ensure that the endotracheal tube 90 can be precisely placed into the glottis of the trachea by reference to the image captured by the bar-shaped camera 10. The handle portion 20a has two convex points 21 individually disposed at the lateral insides of the handle portion 20a for detachably connected with the guiding portion 30b.

As shown in FIG. 8, the guiding portion 30b is detachably connected to the handle portion 20a, and the guiding portion 30b comprises a front end 31, a rear end 32, a tongue piece 35, a barrier 33a and a slot portion 34a. The rear end 32 comprises a connection portion 321 for detachably connecting with the handle portion 20a. The slot portion 34a is situated at the front end 31 of the guiding portion 30b. The tongue piece 35 is extended form the rear end 32 to the front end 31.

The barrier 33a is extended form the rear end 32 to the front end 31 and substantially perpendicular to the tongue piece 35. It is noted that “substantially perpendicular” means that the angle formed by the barrier 33a and the tongue piece 35 is in-between 80^{□} to 100^{□}. The slot portion 34a is connected with the front end 31, thus a restricted passage 39 is formed by the tongue piece 35, the slot portion 34a and the barrier 33a. An open passage 38 is formed by the tongue piece 35 and the barrier 33a; therefore, the endotracheal tube 90 and the bar-shaped camera 10a is movably placed within the open passage 38 and the restricted passage 39. Further, as shown in FIG. 8, the slot portion 34a comprises a main surface portion 341 connected with the barrier 33a and substantially paralleled to the tongue piece 35.

As shown in FIG. 9 and FIG. 10, the surface portion 341 further comprises a hook element 3411 situated at the rear end 32 and paralleled to the barrier 33 for confining the endotracheal tube 90 with the open passage 38. As shown in FIG. 8 to FIG. 10, the connection portion 321 is an accommodation slot with an open end 3211, a closed end 3212 and two engaging holes 3213. As shown in FIG. 10, the barrier 33a is diagonally extended form the closed end 3212 to the front end 31. Because the endotracheal tube 90 naturally curved, it is easier to complete the placement of the endotracheal tube 90 and the bar-shaped camera 10 for entering the restricted passage 39 with a diagonally extend barrier 32. The two engaging holes 3213 engages with the two convex points 21 to complete the connection between the connection portion 321 and the handle portion 20a.

It is noted that, as shown in FIG. 10, the tongue piece 35 is extended form the rear end 32 to the front end 31 along a normal axis Y1, the barrier 33a is extended form the rear end 32 to the front end 31 along an oblique axis Y2, and an included angle θ is formed by the normal axis Y1 and the oblique axis Y2, wherein 10^{□}□θ□40^{□}. As shown in FIG. 11, the barrier 33a is situated underneath the tongue piece 35. The endotracheal tube 90 and the bar-shaped camera 10a are situated within the restricted passage 39 formed by the barrier 33a, the tongue piece 35 and the surface portion 341. It is note that the usage and operating procedure of the tracheal intubation device 1d are the same as those of the tracheal intubation device 1. Therefore, the description is omitted. For such information, please refer to FIG. 2 and FIG. 3.

It must be noted that the above-mentioned embodiments are only for illustration. It is intended that the present invention covers modifications and variations of this invention provided that they fall within the scope of the following claims and their equivalents. Therefore, it will be apparent to those skilled in the art that various modifications can be made to the structure of the present invention without departing from the scope or spirit of the invention.

What is claimed is:

1. A tracheal intubation device for placing an endotracheal tube in a trachea of a patient, the tracheal intubation device comprising:
a bar-shaped camera placed inside an endotracheal tube for capturing an image;
a handle portion;
a display device connected with the handle portion and electrically connected to the bar-shaped camera for displaying the image captured by the bar-shaped camera; and
a guiding portion connected to the handle portion comprising a front end, a rear end, a tongue piece, a barrier and a slot portion, wherein the tongue piece is extended form the rear end to the front end, the barrier is extended form the rear end to the front end and substantially perpendicular to the tongue piece, the slot portion comprises a main surface portion connected with the barrier and substantially paralleled to the tongue piece, the slot portion connected with the front end, thus a restricted passage is formed by the tongue piece, the slot portion and the barrier, an open passage is formed by the tongue piece and the barrier;
whereby, the endotracheal tube and the bar-shaped camera are movably placed within the open passage and the restricted passage.

2. The tracheal intubation device as claimed in claim 1, wherein the tongue piece is extended form the rear end to the front end along a normal axis, the barrier is extended form the rear end to the front end along an oblique axis, and an included angle θ is formed by the normal axis and the oblique axis, wherein 10^{□}□θ□40^{□}.

3. The tracheal intubation device as claimed in claim 2, wherein the surface portion comprises a hook element situated at the rear end and paralleled to the barrier.

4. The tracheal intubation device as claimed in claim 3, wherein the rear end further comprises a connection portion and the guiding portion is detachably connected with the a handle portion via the connection portion.

5. The tracheal intubation device as claimed in claim 4, wherein the connection portion is an accommodation slot with an open end and a closed end, the barrier is diagonally extended form the closed end to the front end.

6. The tracheal intubation device as claimed in claim 5, wherein the tracheal intubation device further comprises a processor, a receiving module, a cell phone module, a memory, and a transmitting module, wherein the processor, the receiving module, the cell phone module, and the memory are separately electrically connected to the display device and the transmitting module is connected to the bar-shaped camera.

7. The tracheal intubation device as claimed in claim 6, wherein the display device and the handle portion are connected.

8. The tracheal intubation device as claimed in claim 1, wherein the surface portion comprises a hook element situated at the rear end and paralleled to the barrier.

9. The tracheal intubation device as claimed in claim 8, wherein the tongue piece is extended form the rear end to the front end along a normal axis, the barrier is extended form the rear end to the front end along an oblique axis, and an included angle θ is formed by the normal axis and the oblique axis, wherein 10^{□}□θ□40^{□}.

10. The tracheal intubation device as claimed in claim 9, wherein the tracheal intubation device further comprises a processor, a receiving module, a cell phone module, a memory, and a transmitting module, wherein the processor, the receiving module, the cell phone module, and the memory are separately electrically connected to the display device and the transmitting module is connected to the bar-shaped camera.

11. The tracheal intubation device as claimed in claim 10, wherein the display device and the handle portion are connected.

12. The tracheal intubation device as claimed in claim 1, wherein the slot portion comprises a restricted part connected with the surface portion and paralleled to the barrier.

13. The tracheal intubation device as claimed in claim 12, wherein the front end further comprises a protrusion part, and the slot portion does not extend to the protrusion part.

14. The tracheal intubation device as claimed in claim 13, wherein the tracheal intubation device further comprises an insertion unit mounted on the bar-shaped camera for placing inside the endotracheal tube.

15. A tracheal intubation device for placing an endotracheal tube in a trachea of a patient, the tracheal intubation device comprising:
a bar-shaped camera placed inside an endotracheal tube for capturing an image, wherein the bar-shaped camera is made of a rigid material that allows the bar-shaped camera to guide the direction of movement of the endotracheal tube while the endotracheal tube is moving;
a handle portion;
a display device electrically connected to the bar-shaped camera for displaying the image captured by the bar-shaped camera; and
a guiding portion connected to the handle portion comprising a front end and a rear end, wherein the rear end of the guiding portion is connected to the handle portion, and the bar-shaped camera and the endotracheal tube are movably situated in the guiding portion; wherein the guiding portion comprises a tongue piece and a barrier; the tongue piece is substantially perpendicular to the barrier and the endotracheal tube and the bar-shaped camera can be accommodated within an area formed by the tongue piece and the barrier.

16. The tracheal intubation device as claimed in claim 15, wherein the bar-shaped camera is detachably connected to the handle portion.
